(19) United States
(12) Patent Application Publication
(10) Pub. No.: US 2024/0400969 A1
(43) Pub. Date: Dec. 5, 2024

(54) AUTOMATED METHOD AND SYSTEM FOR FORMATION OF MESH SUPPORTED TISSUE MEMBRANE

(71) Applicant: DEKA Products Limited Partnership, Manchester, NH (US)

(72) Inventors: Stuart A. Jacobson, Lexington, MA (US); Katie E. Leon, Ashland, MA (US); Hunter A. Higby, Manchester, NH (US); Richard J. Lanigan, Concord, NH (US); Andrew K. Capulli, Auburn, NH (US)

(21) Appl. No.: 18/656,660

(22) Filed: May 7, 2024

Related U.S. Application Data
(60) Provisional application No. 63/505,179, filed on May 31, 2023.

(51) Int. Cl. C12M 1/36; C12M 1/00; C12M 1/12; C12M 1/34; C12N 5/079; G01N 35/10

(52) U.S. Cl. CPC C12M 41/48; C12M 23/38; C12M 23/50; C12M 25/14; C12M 29/20; C12M 37/04; C12M 41/12; C12M 41/36; C12M 41/44; C12N 5/0621; G01N 35/10

(57) ABSTRACT

A system and method for automated production of a viable cell culture on a mesh supported membrane lattice that is suitable for therapeutic implantation in connection with regenerative cell therapy. At least one bioreactor vial is configured to be supported and received by an automated handling and processing system. The bioreactor vial has a reactor well therein into which a mesh-supported submicron parylene-C membrane (MSPM) scaffold is received. Various support fluids are added and subsequently RPE cells are seeded onto the MSPM. The RPE cells form a culture of monolayer of hexagonally shaped RPE cells that is adhered to the MSPM which is suitable for subsequent transplantation into an eye in order to develop in a manner that supports and maintains the photoreceptors of the retina. The system is preferably automated and configured to process multiple bioreactors simultaneously.

### CROSS REFERENCE TO RELATED APPLICATIONS

This application claims priority to U.S. Provisional App. 63/505,179, filed May 31, 2023, the contents of which are incorporated herein by reference.

### BACKGROUND

The present disclosure relates to a method and system for developing cell infused patches for therapeutic implantation. More specifically, the present disclosure relates to a method and system for automated production of cell culture on a mesh supported membrane lattice that is suitable for therapeutic implantation in connection with regenerative cell therapy.

Age-related macular degeneration (AMD) is one of the leading causes of blindness in the elderly. AMD is usually characterized by the degeneration of Bruch's membrane and the resulting dysfunction of retinal pigment epithelial (RPE) cells. As depicted at FIG. 1, the anatomy of an eye 1000 is depicted with an exploded view of the layers at the back of the eye 1010. Bruch's membrane 1020 is a thin structure within the eye (2-4 μm in thickness), comprising the RPE layer 1030, the RPE basement membrane 1040, an inner collagenous zone 1050, elastic fiber bands 1060, an outer collagenous zone 1070 and the choroid basement layer 1080, all located between the RPE cells 1030 and vascular choroid 1090. The Bruch's membrane 1020 is semipermeable and allows the transportations of nutrients and other necessary macromolecules from underlying blood vessels in the choroid 1090 to retina. Directly on Bruch's membrane is a monolayer of hexagonally shaped RPE cells that support and maintain the photoreceptors of the retina. One theory postulates that, in AMD, RPE cells stop degrading the waste products of photoreceptors properly, leading to the accumulation of wastes in Bruch's membrane. As a result, Bruch's membrane becomes thickened and its composition changes, lowering the permeability to nutrients and macromolecules, which causes dysfunction of RPE cells and loss of photoreceptors, ultimately resulting in severe vision loss.

In the prior art attempts have been made to replace the diseased RPE cells as a potential treatment of AMD. However, attempts to directly transplant healthy RPE cells were unsuccessful because the transplanted cells usually had poor adherence on the native Bruch's membrane and failed to form a monolayer on it. More promising alternative methods involve transplanting in vitro cultured RPE cells as a preformed monolayer on a substitute substrate. In order to be a functional replacement substrate, such an artificial Bruch's membrane must be biocompatible and possess similar permeability to human Bruch's membrane. Moreover, it must also support the attachment and growth of RPE cells that are similar in character to the existing RPE cells in vivo. Substrates made of various materials have been studied as artificial Bruch's membranes, such as poly (DL-lactic-co-glycolic acid) (PLGA), polydimethylsiloxane (PDMS), poly (methyl methacrylate) (PMMA), human lens capsule, collagen type I, elastin-like recombinamers, and other synthetic degradable or non-degradable polymers.

Although a number of substrates have been demonstrated to be potential candidates as an artificial Bruch's membrane, many of them still have shortcomings. Some biological tissues, such as lens capsule and collagen I, are difficult to handle. For example, it is difficult to obtain a large, continuous piece of lens capsule without holes or tears. Collagen film has to be prepared on a Teflon support and this Teflon support which needs to be removed before implantation, thereby increasing the complexity of handling and may cause contamination. For the synthetic degradable materials, it is still unclear whether their breakdown products and removal have influences on the already compromised retina-choroid complex. Although PDMS with proper surface treatment could support RPE cell growth, the ultrathin PDMS with comparable thickness to Bruch's membrane is difficult to make.

Another preferred substrate for creation of transplantable artificial Bruch's membrane is parylene-C. As a USP class VI biocompatible polymer, parylene-C has found numerous biomedical applications. Due to its good mechanical strength, biostability, barrier properties and chemical inertness, parylene-C is usually adopted in implantable devices which require isolation from moisture, chemicals and corrosive body fluids and tissues. However, it has been found that when the thickness of parylene-C is reduced to the submicron range, it becomes semipermeable to molecules with certain molecular weights (MW), making it suitable as a substrate for the growth of an artificial Bruch's membrane. Perfusion cell viability tests further confirm that 0.30 μm parylene-C is able to support sufficient nutrients transportation. To enhance the mechanical support of the ultrathin parylene-C, a mesh-supported submicron parylene-C membrane (MSPM) is employed which was demonstrated to support RPE cell growth with in vivo-like characteristics.

To be effective for the therapy of age-related macular degeneration (AMD), any artificial Bruch's membrane must first satisfy two important requirements. First, it should be as permeable as healthy human Bruch's membrane to support nutrients transportation. Secondly, it should be able to support the adherence and proliferation of retinal pigment epithelial (RPE) cells with in vivo-like morphologies and functions. Although parylene-C is widely used as a barrier layer in many biomedical applications, it is found that parylene-C membranes with submicron thickness are semipermeable to macromolecules. Parylene-C, with a thickness on the order of 0.15 μm-0.30 μm, has similar permeability to healthy human Bruch's membranes. Blind-well perfusion cell viability experiments further demonstrate that nutrients and macromolecules can diffuse across 0.30 μm parylene-C to nourish the cells. A mesh-supported submicron parylene-C membrane (MSPM) structure further enhances the mechanical strength of the substrate. An in vitro cell culture on the MSPM (with 0.30 μm ultrathin parylene-C) shows that H9-RPE cells are able to adhere, proliferate, form epithelial monolayer with tight intracellular junctions, and become well-polarized with microvilli, exhibiting similar characteristics to RPE cells in vivo.

Since the submicron parylene-C itself lacks enough mechanical strength, a MSPM structure is employed. One potential MSPM fabrication process is illustrated at FIG. 2. At Step (a) the process is started by depositing a 6 μm thick structural frame of parylene-C 2010 on a base of hexamethyldisilazane (HMDS) treated silicon 2020. At Step (b) Aluminum 2030 is then deposited over the parylene-C 2010 as an etching mask followed by a photo resistive spin-coating 2040. At Step (c) lithography, wet etching of the aluminum, and reactive ion etching (RIE) with oxygen plasma. At Step (d) diluted hydrofluoric acid (HF) is used to clean the residues inside the holes revealing the patterned parylene-C 2010 having circular through holes as a mesh frame. At Step (c) a submicron parylene-C film (e.g., 0.15 μm-0.80 μm) is then deposited on the parylene-C mesh frame. Step (f) shows a second lithography to cover the whole device with photoresist and the contour of the individual parylene-C membranes are formed by etching away ultrathin parylene-C in the undesirable regions leaving parylene-C 2010 on a base of hexamethyldisilazane (HMDS) treated silicon 2020 at Step (g). Finally, at Step (h), the whole membrane is peeled off and flipped over. Both sides of the membrane are treated with low power oxygen plasma (power: 50 W, chamber pressure: 200 mTorr, duration: 1 min) for better cell adherence.

FIGS. 3A, 3B and 3C show SEM images of the front, flat side (FIG. 3A), the back side, exhibiting openings (FIG. 3B) and a cross-section (FIG. 3C) of the completed MSPM 3000. A magnified cross-sectional cut of the MSPM 3000 shown at FIG. 3C shows the 6 μm thick structural frame 3010 and the 0.15 μm-0.80 μm submicron permeable film 3020 disposed in the openings 3030 of the structural frame 3010.

The present technique employed for forming a viable RPE culture on the MSPM involves placing a plurality of sheets, each on the order of on the order of 3.5 mm by 6.25 mm, into a culture wells on a well plate, applying vitronectin, a glycosaminoglycan protein that promotes cell adhesion and cell spread, over-seeding the entire sheet of MSPM with RPE cells, and applying growth media to the over-seeded RPE cells to produce a viable cell culture over the entire sheet of the MSPM. The sheets of MSPM containing the RPE cultures are then individually loaded onto cryovials for transport and ultimate transplantation.

The difficulty in the prior art process of preparation, culture and transport of the MSPM patches is that the process is nearly entirely manual in nature. This requires larger quantities of viable RPE cells, growth media and vitronectin for seeding of the MSPM than would be actually necessary in an efficient process. Further, the process of cutting the MSPM sheets to create a handling region with RPE cultures thereon is difficult in that the cuts must be precise to prevent the formation of burrs on the cut edges of the individual patches as well as to avoid cutting of individual RPE cells that results in the initiation of a wound healing process. Still further, the subsequent handling of the MSPM patches can lead to damage, crumpling or folding of the MSPM patches resulting in increased defect rates. Finally, the MSPM patches have in intended orientation, wherein the RPE culture is to be grown on the front, flat side of the MSPM such that care must be taken to insure proper orientation of the base MSPM prior to seeding of the RPE cells thereon.

Accordingly, there is a need for a method and system for automated production of a mesh supported membrane lattice for supporting cultured cells that is suitable for therapeutic implantation in connection with regenerative cell therapy. There is a further need for a method and system that provides maximum throughput and efficient production of a mesh supported membrane lattice for supporting cultured cells that is suitable for therapeutic implantation in connection with regenerative cell therapy. There is a still further need for a method and system for automated production of a mesh supported membrane lattice for supporting cultured cells that is suitable for therapeutic implantation in connection with regenerative cell therapy that reduces damage as well as potential contamination to the tissue culture during growth, handling, transport and preparation for transplantation. There is a still further need for a method and system for automated production of a mesh supported membrane lattice for supporting cultured cells that is suitable for therapeutic implantation in connection with regenerative cell therapy that improves the efficiency with which the quantities of viable RPE cells, growth media and vitronectin are consumed in producing the completed MSPM scaffolding supporting a culture of monolayer of hexagonally shaped RPE cells.

### SUMMARY

In accordance with some configurations, the present disclosure provides most generally a method and system for automated production of cultured cells on a mesh supported membrane lattice that is suitable for therapeutic implantation in connection with regenerative cell therapy. In one embodiment at least one bioreactor vial is provided that is configured to be supported and received by an automated handling and processing system. Each bioreactor vial has an individual reactor well into which an MSPM scaffold is received. Various support fluids are added and subsequently RPE cells are seeded onto the MSPM. The RPE cells form a culture of monolayer of RPE cells that is adhered to the MSPM which is suitable for subsequent transplantation into an eye in order to develop in a manner that supports and maintains the photoreceptors of the retina.

In another configuration, the present disclosure teaches a method and system that provides maximum throughput and efficient production of a mesh supported membrane lattice for supporting cultured cells that is suitable for therapeutic implantation in connection with regenerative cell therapy. One or more bioreactor vials are provided wherein each bioreactor vial is configured to be supported and received by an automated handling and processing system. The bioreactor vial has a reactor well therein into which an MSPM scaffold is received. Various support fluids are added and subsequently RPE cells are seeded onto the MSPM. The RPE cells form a culture of monolayer of hexagonally shaped RPE cells that is adhered to the MSPM which is suitable for subsequent transplantation into an eye in order to develop in a manner that supports and maintains the photoreceptors of the retina.

In a further configuration, the present disclosure teaches a method and system for automated production of a mesh supported membrane lattice for supporting cultured cells that is suitable for therapeutic implantation in connection with regenerative cell therapy. A plurality of MSPM scaffolds are individually positioned in a reactor well within each of a plurality of bioreactor vials, the bioreactor wells each configured to securely receive and retain the MSPM scaffold received therein. A fluid delivery system transfers required process fluids into and out of the reactor well in a manner that reduces damage as well as potential contamination to the tissue culture during growth, handling, transport and preparation for transplantation.

In still a further configuration, the present disclosure provides a method and system for automated production of a mesh supported membrane lattice that supports cultured cells that is suitable for therapeutic implantation in connection with regenerative cell therapy, wherein a reactor well in each of a plurality of bioreactor vials is sized and shaped in a manner that improves the efficiency with which the quantities of viable RPE cells, growth media and vitronectin are consumed in producing the completed MSPM patches.

In yet a further configuration, the present disclosure teaches a method and system for automated production of a mesh supported membrane lattice for supporting cultured cells that is suitable for therapeutic implantation in connection with regenerative cell therapy wherein a shuttle system having a plurality of bioreactor stations wherein each bioreactor station is configured to receive and retain a bioreactor vial therein. The shuttle system is movable in a manner that each station is moved to position the bioreactor vial adjacent to one or more material delivery stations that serve to position an MSPM scaffold in each of the reactor wells within each of a plurality of bioreactor vials, deliver and remove various fluids, seed cells onto the MSPM scaffolds, deliver cryogenic preservative fluid and/or cap or seal the bioreactor vials in a manner that reduces damage as well as potential contamination to the tissue culture during growth, handling, transport and preparation for transplantation.

In some configurations the bioreactor vials are sealed and accessed via ports therein. In some configurations, the bioreactor vials have a displaceable cap that remains in an open position until the final step in processing.

In some configurations, the reactor well in each of the bioreactor vials include elements that direct and control fluid movement within the reactor well to prevent dislodging, floating, crumpling, creasing and/or inversion of the MSPM scaffold contained therein.

In some configurations, the shuttle system is in a closed environment.

In some configurations, the shuttle system has multiple tiers, each tier including a plurality of bioreactor stations thereon.

In other configurations, a camera is included to provide visualization of the monolayer of cultured cells on the MSPM scaffold contained within the reactor well of the bioreactor vials.

In a further configuration in accordance with the present disclosure, a method is taught for automated production of a mesh supported membrane lattice for supporting cultured cells that is suitable for therapeutic implantation in connection with regenerative cell therapy. At least one bioreactor vial is provided that is configured to be supported and received by an automated handling and processing system is provided. An MSPM scaffold is positioned in a reactor well within the bioreactor vial. Support fluids are added and subsequently RPE cells are seeded onto the MSPM by a fluid handling system. The RPE cells are cultured to form a monolayer of hexagonally shaped RPE cells that is adhered to the MSPM which is suitable for subsequent transplantation into an eye in order to develop in a manner that supports and maintains the photoreceptors of the retina.

In a further configuration, support fluids may include, a buffer solution for maintaining cell culture media in the physiological range of 7.2-7.6, such as Dulbecco's Phosphate Buffered Saline (DPBS). Support fluids may further include, a solution of viable RPE cells, growth media, vitronectin, cryo preservative and combinations thereof.

In another configuration, the present disclosure teaches a method and system that provides maximum throughput and efficient production of a mesh supported membrane lattice for supporting cultured cells that is suitable for therapeutic implantation in connection with regenerative cell therapy. One or more bioreactor vials are provided wherein each bioreactor vial is configured to be supported and received by an automated handling and processing system. The bioreactor vial has a reactor well therein into which an MSPM scaffold is received. Various support fluids are added and subsequently RPE cells are seeded onto the MSPM. The RPE cells form a monolayer of hexagonally shaped RPE cells that is adhered to the MSPM which is suitable for subsequent transplantation into an eye in order to develop in a manner that supports and maintains the photoreceptors of the retina.

In a further configuration in accordance with the present disclosure, a method is taught for automated production of a mesh supported membrane lattice for supporting cultured cells that is suitable for therapeutic implantation in connection with regenerative cell therapy wherein a plurality of MSPM scaffolds are individually positioned in a reactor well within each of a plurality of bioreactor vials. Required process fluids are transferred into and out of the reactor well by a fluid delivery system in a manner that reduces damage as well as potential contamination to the tissue culture during growth, handling, transport and preparation for transplantation.

In yet a further configuration, the present disclosure teaches a method for automated production of a mesh supported membrane lattice for supporting cultured cells that is suitable for therapeutic implantation in connection with regenerative cell therapy wherein plurality of bioreactor vials is individually placed in a shuttle system having a plurality of bioreactor stations, wherein each bioreactor station is configured to receive and retain a bioreactor vial therein. Operating the shuttle system to position each bioreactor vial adjacent one or more material delivery stations that serve to position an MSPM scaffold in each of the reactor well within each of a plurality of bioreactor vials, deliver and remove various fluids, seed cells onto the MSPM scaffolds, deliver cryogenic preservative fluid and/or cap or seal the bioreactor vials in a manner that reduces damage as well as potential contamination to the tissue culture during growth, handling, transport and preparation for transplantation.

In some configurations the method includes a step wherein the bioreactor vials are accessed via ports that are sealed at the end of the process. In other aspects, the method includes a step wherein the bioreactor vials are accessed via an opening at a top thereof and subsequently sealed by a displaceable cap that remains in an open position until the final step in processing.

In other configurations, the RPE cell culture within the reactor well is imaged by a camera that provides visualization of the MSPM scaffold contained within the reactor well of the bioreactor vials.

Accordingly, it is an object of the disclosure to provide a method and system for automated production of a mesh supported membrane lattice for supporting cultured cells that is suitable for therapeutic implantation in connection with regenerative cell therapy. It is a further object of the present disclosure to provide a method and system that provides maximum throughput and efficient production of a mesh supported membrane lattice for supporting cultured cells that is suitable for therapeutic implantation in connection with regenerative cell therapy. It is still a further object of the present disclosure to provide a method and system for automated production of a mesh supported membrane lattice for supporting cultured cells that is suitable for therapeutic implantation in connection with regenerative cell therapy that reduces damage as well as potential contamination to the tissue culture during growth, handling, transport and preparation for transplantation. It is a still further object of the disclosure to provide a method and system for automated production of a mesh supported membrane lattice for supporting cultured cells that is suitable for therapeutic implantation in connection with regenerative cell therapy that improves the efficiency with which the quantities of viable RPE cells, growth media and vitronectin are consumed in producing the completed MSPM scaffolding supporting a culture of monolayer of hexagonally shaped RPE cells.

In some aspects of the method and system of the present disclosure, some components are single use, disposable parts such as, bioreactor vials, syringes, tubing sets and waste containers. Other components are durable reusable components such as the shuttle system, an enclosure, pumping subsystem and the imaging subsystem. The use of a combination of disposable and durable parts ensures sterility and maximizes product yield during implementation of the automated process.

These together with other objects of the disclosure, along with various features of novelty which characterize the method and system of the present disclosure, are pointed out with particularity in the claims annexed hereto and forming a part of this disclosure. For a better understanding of the method and system of the disclosure, its operating advantages and the specific objects attained by its uses, reference should be had to the accompanying drawings and descriptive matter in which there are illustrated various embodiments of the method and system disclosed herein.

### BRIEF DESCRIPTION OF THE DRAWINGS

The foregoing features of the disclosure will be more readily understood by reference to the following description, taken with reference to the accompanying drawings, in which:

FIG. 1 is a depiction of the anatomy of an eye with an expanded view of the structures and layers at the posterior of the eye;

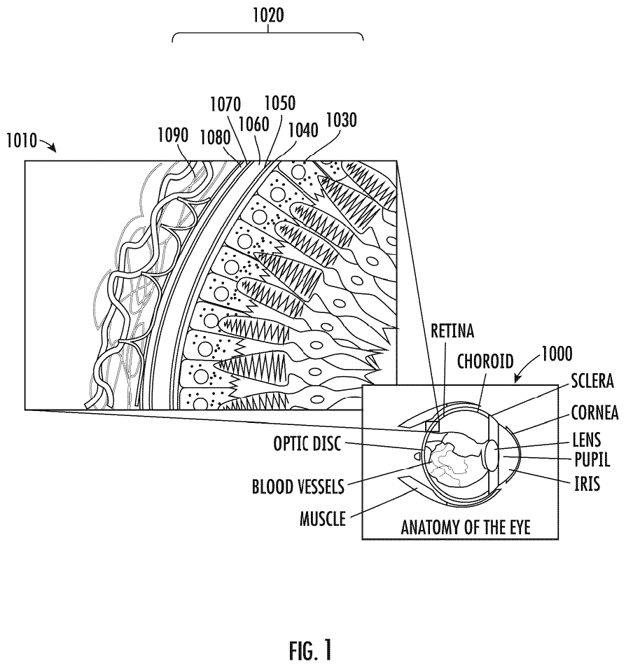

FIG. 2 is an illustration of a process for forming a mesh-supported submicron parylene-C membrane (MSPM) structure as a scaffold for use in connection with the method and system of the present disclosure;

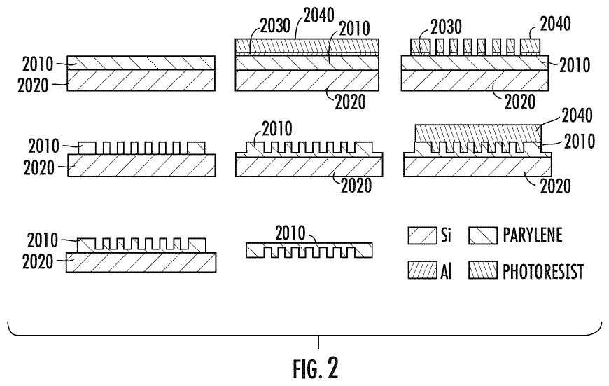

FIGS. 3A-3C are scanning electron microscope (SEM) images of the front, flat side (FIG. 3A), the back side, exhibiting openings (FIG. 3B) and a cross-section (FIG. 3C) of a completed MSPM;

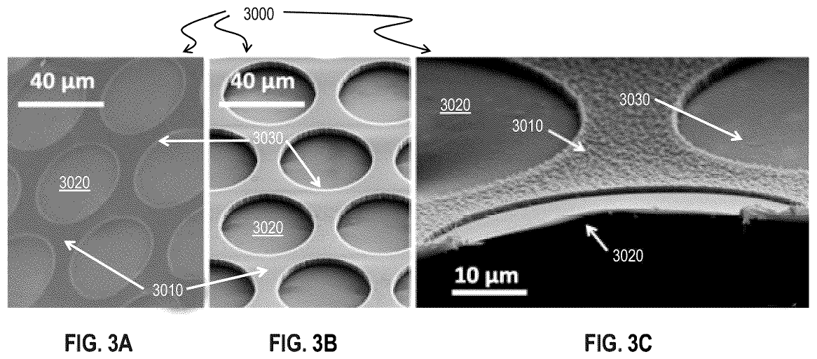

FIG. 4 depicts an illustration of a system in accordance with the present disclosure;

FIG. 5 depicts an automated bioreactor vial handling system in accordance with the present disclosure;

FIG. 6 is a partial exploded view of the top of the automated handling system of the present disclosure;

FIG. 7 is a partial exploded view of the bottom of the automated handling system of the present disclosure;

FIG. 8 is a partial top view of one configuration of the automated handling system of the present disclosure;

FIG. 9 is a view of one of the actuator arms in accordance with one configuration of the automated handling system of the present disclosure;

FIG. 10 is a cross-sectional view of one of the actuator arms taken along line 10-10 of FIG. 9;

FIGS. 11A-11C show one configuration of a bioreactor vial for use in connection with the automated handling system of the present disclosure;

FIGS. 12A-12D show an alternate configuration of a bioreactor vial for use in connection with the automated handling system of the present disclosure;

FIGS. 13A-13C show another alternate configuration of a bioreactor vial for use in connection with the automated handling system of the present disclosure;

FIG. 14 shows a configuration of an imaging system for use in connection with the automated handling system of the present disclosure;

FIG. 15 shows a configuration of a fluid handling system for use in connection with the automated handling system of the present disclosure;

FIGS. 16A-16B show a configuration of a control system for use in connection with the automated handling system of the present disclosure;

FIG. 17 is a partial top view of an alternate configuration of the automated handling system of the present disclosure;

FIGS. 18A-18B show an alternate configuration of a bioreactor vial for use in connection with the automated handling system depicted at FIG. 17 of the present disclosure;

FIG. 19 is an alternate partial top view of the automated handling system depicted at FIG. 17 of the present disclosure;

FIG. 20 provides a schematic flow diagram of a method on accordance with the teachings of the present disclosure; and

FIG. 21 provides a schematic diagram of a thermal management system and method.

### DETAILED DESCRIPTION

The system of the present teachings for maintaining, assessing, maturing, and rehabilitating tissue is described in detail herein. Specifically, the system and method of the present teachings is configured to allow for real-time assessment of the tissue, using that assessment to continuously maintain the health of the tissue.

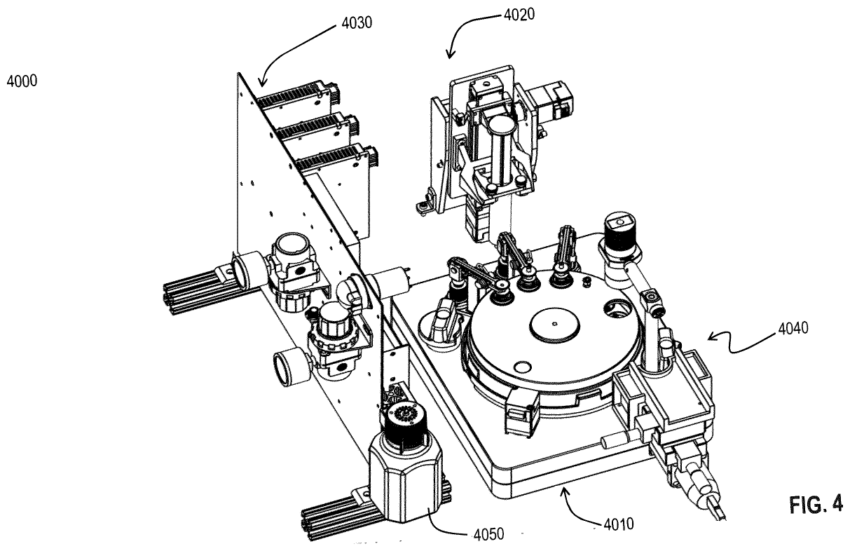

The present disclosure provides, most generally, a method and system for automated production of a mesh supported membrane lattice that supports cultured cells and is suitable for therapeutic implantation in connection with regenerative cell therapy. As can be seen in FIG. 4, the system 4000 generally includes an automated handling system 4010 that receives and retains the at least one bioreactor vial, a fluid delivery system 4020 that adds and/or removes support fluids to/from the at least one bioreactor vial, a controller 4030 for directing and controlling the operation of the system 4000 and its various components, an imaging system 4040 for observing and monitoring the culture activity within the at least one bioreactor vial, and a waste container 4050 for capturing any fluidic byproducts from the method and system of the present disclosure. In addition, the system may further include other operational components such as a thermal control system, such as for example an incubator, to maintain a predetermined temperature in the at least one bioreactor vial, an enclosure to maintain a sealed environment around at least the at least one bioreactor vial.

The system of the present teachings includes, but is not limited to including, a disposable set of components and a durable set of components. The disposable components include, but are not limited to including, at least one bioreactor vial, at least one syringe received in a syringe pump and at least one bioreactor interface tube that are included as part of the fluid delivery system, at least one waste container, and tubing that fluidically connects the various components. The durable components include, but are not limited to including, a base of the automated handling system, at least one shuttle carrier for receiving and handling at least one bioreactor vial, arms that receive support bioreactor interface tubes, a controller, and an imaging system.

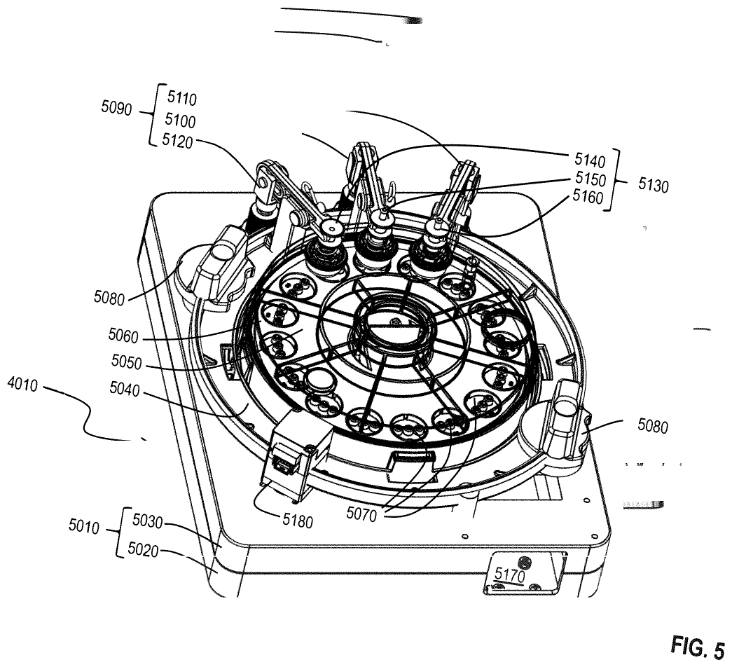

Turning now to FIG. 5, the automated handling system 4010 is illustrated in greater detail. A base 5010 is provided comprising an upper base 5030 and a lower base 5020 that are separable from one another to allow access and/or servicing of components contained therein. The base supports the operational components of the automated handling system 4010 and maintains their relative positioning during implementation of the method of the present disclosure. A shuttle table 5040 can be seen residing on a top surface of the upper base 5030. The shuttle table 5040 is configured to support and guide the motion of a shuttle carrier 5050 received in a recess formed therein. The shuttle table 5040 has an upper rim that is configured to mate with lid 5060 wherein the shuttle table 5040 and lid cooperate to form a sealed environment in which the shuttle carrier 5050 and at least one bioreactor vial 5070 are contained during the method of the present disclosure. Shuttle table locks 5080 shown on the upper base 5030 are configured to releasably engage the shuttle table 5040 and retain the shuttle table 5040 in in position on the upper base 5030.

Spring biased actuator arms 5090 include at least one input arm 5110. Further, other configurations may also include a spring biased drain arm 5100. Still further, in some embodiments, the system may include a spring biased capping arm 5120. The actuator arms 5090 have a normally retracted position such that they are biased to reside in an upward, retracted position. The actuator arms 5090 engage with interface tubes 5130, wherein there is an inlet tube 5140 and a drain tube 5150. In some configurations interface tubes 5130 may also include a capping tube 5160. The operation and function of the actuator arms 5090 and interface tubes 5130 will be described in greater detail below.

Still referring to FIG. 5, an imaging system opening 5170 is provided in the base 5010 that allows installation of the imaging system 4040 such that it can be positioned above and below the at least one bioreactor vial 5070 so as to simultaneously illuminate and image the cell culture within the bioreactor vial 5070. Further, a stepper motor and controller 5180 is provided that controls and advances the shuttle carrier 5050 as will be described in detail below.

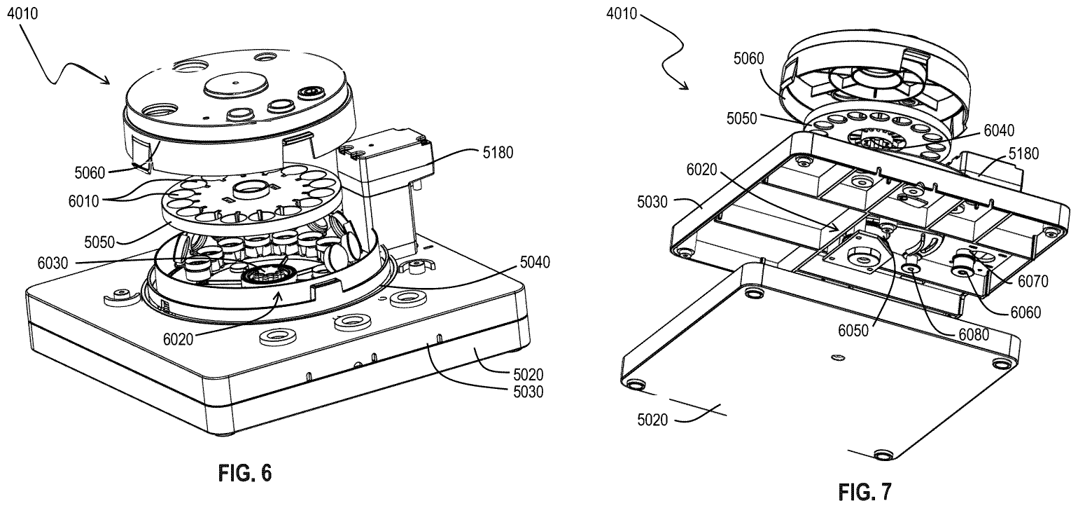

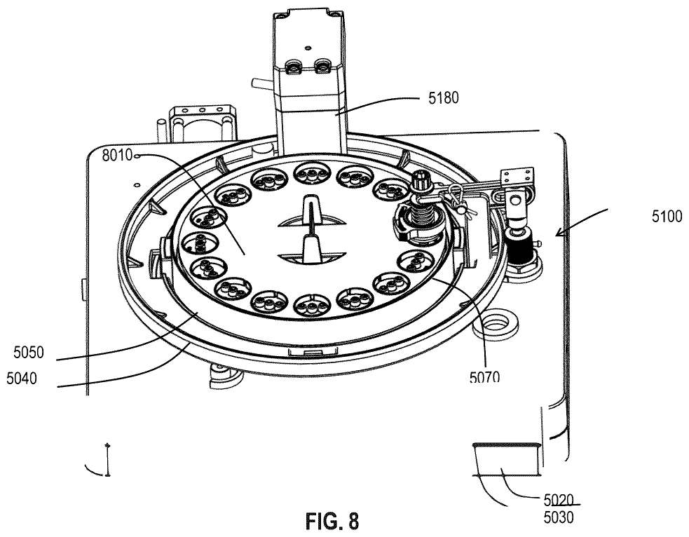

Now discussing FIGS. 6 and 7 in combination, an exploded top and bottom view of the automated handling system 4010 is shown. In some configurations, the shuttle carrier 5050 may include a plurality of bioreactor wells 6010 therein that are each configured to receive and support one of a plurality of bioreactor vials 5070 therein. As stated above, the shuttle carrier 5050 is received within the shuttle table 5040 such that the shuttle table 5040 guides the motion of the shuttle carrier 5050. It should be appreciated that while a round shuttle carrier 5050 and a corresponding round shuttle table is shown herein, such that a motion of the shuttle carrier is rotational, such disclosure is not intended to be limiting on the system in that a linear or rectangular motion also falls within the scope of the disclosure. A spindle 6020 can be seen centrally located in the shuttle table 5040 and is rotationally received and supported therein. The top of the spindle 6020 has a toothed formation 6030 formed thereon that releasably engages with a corresponding mating formation 6040 centrally located in the bottom of the shuttle carrier 5050. By engaging with the spindle 6020 in this manner, rotation of the spindle 6030 causes corresponding rotator of the shuttle carrier 5050. Similarly, the spindle 6030 could be located on the periphery of the shuttle table 5040 and the mating formation 6040 would be configured as a corresponding ring gear on the bottom surface of the shuttle carrier 5050. Still further, if the configuration is linear, a linear rack arrangement may be provided on the bottom surface of the shuttle carrier 5050 such that rotation of the spindle 6020 causes linear translation of the shuttle carrier 5050.

The bottom of the spindle 6020 includes a pulley 6050 thereon to engage via belt, chain, direct drive or any other engagement means with a corresponding pulley 6060 on the drive shaft 6070 of the stepper motor 5180 such that rotation of the stepper motor 5180 drive shaft 6070 causes rotation of the spindle 6020 and in turn movement of the shuttle carrier 5050. In some configurations a tensioner assembly 6080 may be provided to tension the connection means between the two pulleys 6050 and 6060 and provide reliable and accurate motion indexing regarding the position of the shuttle carrier 5050.

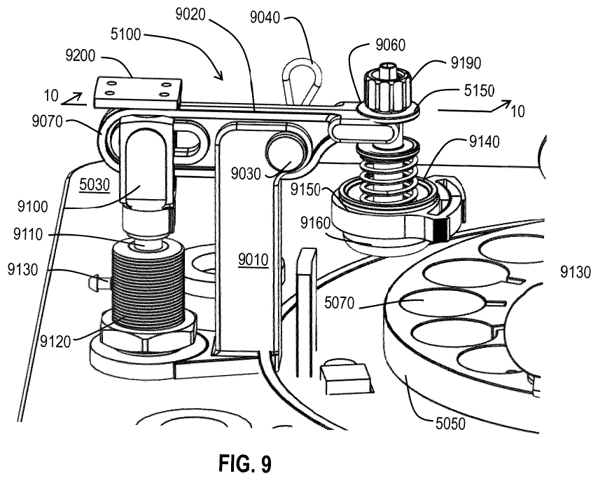

FIG. 8 provides an alternate top view of the automated handling system 4010 with the lid being removed to expose a tray retainer 8010. The tray retainer 8010 is positioned over the at least one bioreactor vial 5070 residing within the shuttle carrier 5050 to maintain the bioreactor vials 5070 in their positions within the shuttle carrier 5050.

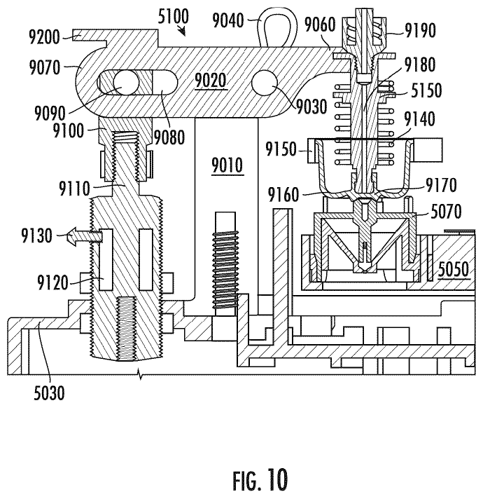

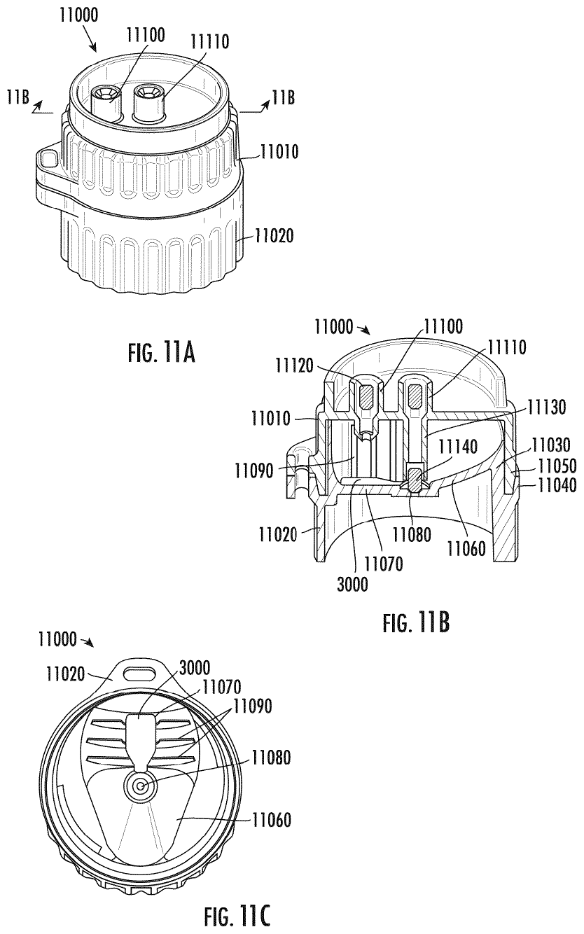

Turning now to FIGS. 9 and 10, an expanded view is provided to illustrate one of the actuator arms 5100. In particular, the drain actuator arm 5100 is shown and the plugging actuator arm 5120 and input actuator arm 5110 have been removed for clarity of illustration. While functioning of the drain actuator arm 5100 will be described, the operational functionality and the manner in which all of the actuator arms 5090 engage with bioreactor vials 5070 is similar. The actuator arms 5090 as illustrated generally by drain actuator arm 5100 each include a vertical support 9010 fastened to the upper base 5030 to provide a firm support, predictable motion and repeatable positioning of the actuator arm. A rocker 9020 is pivotally attached to a top end of the vertical support 9010 by a pin 9030 and retainer clip 9040 in a manner that allows easy removal and replacement of the rocker 9020 as needed when assembling or servicing the system of the present disclosure. Rocker 9020 has a first end 9060 that is configured to engage with a top end of interface tube 5150. The opposing second end 9070 of the rocker 9020 has a slotted opening 9080 therein that receives and engages with pin 9090 extending through actuator rod end 9100. Actuator rod end 9100 is engaged with an actuator rod 9110 extending from and operably coupled into an air actuated piston 9120. Application of control air flow at air inlet 9130 causes vertical displacement of the actuator rod 9110 and actuator rod end 9100 which in turn causes a rotational motion of the rocker 9020 about the pin 9030.

A top end of interface tube 5150 engages with the first end 9060 of rocker arm 9020 while a second opposing end of interface tube 5150 is configured and arranged to engage with injection seal 9160. Injection seal 9160 is a flexible, deformable seal that is retained to the lid 5060 via retainer clip 9150. Spring 9040 spring biases interface tube 5150 upwardly relative to lid 5060 and the bioreactor vial 5070. Downward pressure exerted by rocker 9020 on the interface tube 5150 compresses spring 9140 against the lid 5060 causing interface tube 5150 to move downwardly in a linear fashion thereby extending the injection seal 9160 such that a seat 9170 seals against an access port on the top of the bioreactor vial 5070. In this position, lumen 9180 extending through interface tube 5150 and injection seal 9160 allows the system to interact, as will be described in detail below, with the access ports on the top of bioreactor vial 5070. Interface as between the interface tube 5150 and the fluid delivery system is provided by tubing that engages with the top end of interface tube 5150 cither directly or by any known connector known in the art, such as a luer lock connector 9190 which is illustrated but not intended to be limiting on the present disclosure. Further, a mount 9200 is provided on the top of rocker 9020 to allow for tubing management as tubing extends from the interface tube 5150 back to the fluid delivery system.

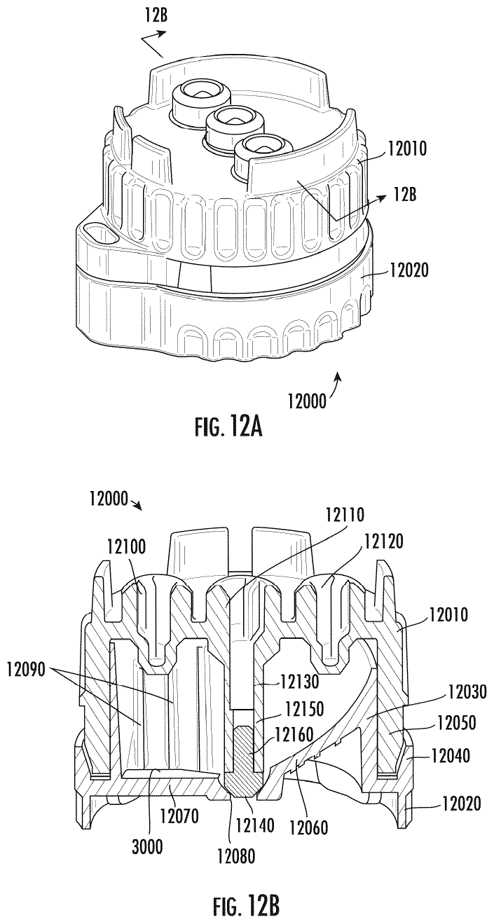

The method and system of the present disclosure is configured and arranged to develop a culture of RPE cells on an MSPM scaffold in an automated manner to reduce handling of the fragile MSPM scaffold as well as to maximize uniform culture growth and minimize risk of contamination throughout the entire process. The components discussed above are provided to receive and support a plurality of individual, self-contained bioreactor vials 5070 throughout the entire process from seeding of RPE cells onto the MSPM scaffold, through culture growth up and until the culture on the MSPM scaffold is prepared for transplantation. In this regard, FIGS. 11A-11C provide an illustration of one configuration of a bioreactor vial 11000 suitable for use in accordance with the teachings herein. The bioreactor 11000 comprises a top shell 11010 and a bottom shell 11020. The bottom shell 11020 includes an upwardly extending side wall 11030 and an upwardly extending retaining wall 11040 that cooperate to from a gap there between that receive and seal against a bottom edge of a downwardly extending sidewall 11050 on the top shell 11010 when the top shell 11010 and bottom shell 11020 are received in assembled relation with one another. A bottom wall 11060 is shaped to have a downwardly tapered shape to urge fluids contained or deposited into the bioreactor vial 11000 to flow into and collect in the reactor well 11070 and around the vial drain 11080. Reactor well 11070 is configured to have a size and shape to closely match the size and shape of MSPM scaffolding 3000 received therein. Fins 11090 rise from the bottom wall 11060 adjacent the reactor well 11070. The fins 11090 are fluid-dynamically engineered so as to retain small amounts of fluid trapped there between when fluid is drawn out of the bioreactor vial 11000 to allow enough fluid to remain and drain back into the reactor well 11070 to ensure that the MSPM 3000 does not dry out during the various stages of the process disclosed herein.

The top shell 11010 includes an inlet port 11100 and a drain port 11110 extending there through. While plugs 11120 are shown, during the process plugs are not seated in a position that blocks the ports until the end of the process as will be described in detail below. As described above, when fluid is added to the bioreactor vial 11000, the interface tube 5150 is depressed such that the injection seal 9160 is seated against the inlet port 11100 and fluid is then pumped via lumen 9180 into the bioreactor vial 11000. The drain port 11110 includes a drain tube 11130 that extends to the bottom wall 11060 of the bioreactor vial 11000. A drain plug 11140 resides at the bottom of the drain tube 11130 and is urged against the vial drain 11080. Channels around a stem of the drain plug 11140 insure that when the interface tube 5150 is depressed such that the injection seal 9160 is seated against the drain port 11110 and a vacuum is applied via lumen 9180 fluid is drawn out of the bioreactor vial 11000. In preparation for transplantation, the user inserts a pin into vial drain 11080 to urge drain plug 11140 upwardly into the end of drain tube 11130 thereby allowing preservative fluid contained within the bioreactor vial 11000 to drain therefrom.

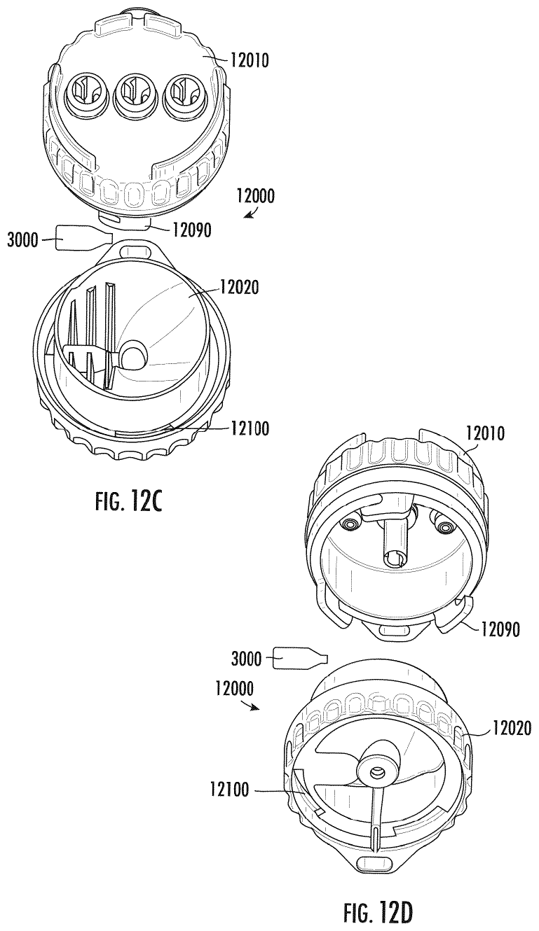

FIGS. 12A-12D provide an illustration of an alternate configuration of a bioreactor vial 12000 suitable for use in accordance with the teachings herein. The bioreactor 12000 comprises a top shell 12010 and a bottom shell 12020. The bottom shell 12020 includes an upwardly extending side wall 12030 and an upwardly extending retaining wall 12040 that cooperate to from a gap there between that receive and seal against a bottom edge of a downwardly extending sidewall 12050 on the top shell 12010 when the top shell 12010 and bottom shell 12020 are received in assembled relation with one another. A bottom wall 12060 is shaped to have a downwardly tapered shape to urge fluids contained or deposited into the bioreactor vial 12000 to flow into and collect in the reactor well 12070 and around the vial drain 12080. Reactor well 12070 is configured to have a size and shape to closely match the size and shape of MSPM scaffolding 3000 received therein. Fins 12090 rise from the bottom wall 12060 adjacent the reactor well 12070. The fins 12090 are fluid-dynamically engineered so as to retain small amounts of fluid trapped there between when fluid is drawn out of the bioreactor vial 12000 to allow enough fluid to remain and drain back into the reactor well 12070 to ensure that the MSPM 3000 does not dry out during the various stages of the process disclosed herein. Further, the fins 12090 also limit the movement of the MSPM 3000 as within the reactor well 12070.

The top shell 12010 includes an inlet port 12100, a drain port 12110 and an air relief port 12120 extending there through. As described above, when fluid is added to the bioreactor vial 12000, the interface tube 5150 is depressed such that the injection seal 9160 is seated against the inlet port 12100 and fluid is then pumped via lumen 9180 into the bioreactor vial 12000. The drain port 12110 includes a drain tube 12130 that extends to the bottom wall 12060 of the bioreactor vial 11000. A drain plug 12140 resides at the bottom of the drain tube 12130 and is urged against the vial drain 12080. Channels 12150 around a stem 12160 of the drain plug 12140 ensure that when the interface tube 5150 is depressed such that the injection seal 9160 is seated against the drain port 12110 and a vacuum is applied via lumen 9180 fluid is drawn out of the bioreactor vial 12000. In preparation for transplantation, the user removes the top shell 12010 which also removes the drain tube 12130 and drain plug 12140 thereby allowing preservative fluid contained within the bioreactor vial 12000 to drain therefrom. As can best be seen in FIGS. 12C and 12D, lock tabs 12090 extend from a bottom edge of top shell 12010 and locking slots 12100 are formed in the recess along the lower portion of the bottom shell 12020. When bottom shell 12020 and top shell 12010 are in mated relation and rotated relative to one another locking tabs 12090 are inserted into locking slots 12100 such that the engage with one another and lock bottom shell 12020 and top shell 12010 in engagement with one another. In this manner, each bioreactor vial is individually locked and scaled to maintain their own sterility.

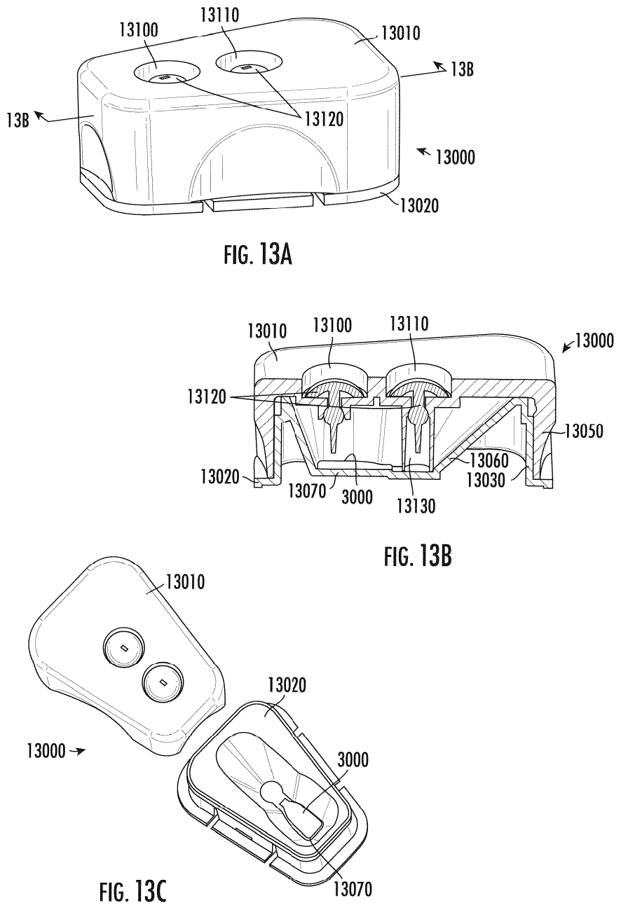

FIGS. 13A-13C provide an illustration of a further alternate configuration of a bioreactor vial 13000 suitable for use in accordance with the teachings herein. The bioreactor 13000 comprises a top shell 13010 and a bottom shell 13020. The bottom shell 13020 includes an upwardly extending side wall 13030 that receives and seal against a bottom edge of a downwardly extending sidewall 13050 on the top shell 13010 when the top shell 13010 and bottom shell 13020 are received in assembled relation with one another. A bottom wall 13060 is shaped to have a downwardly tapered shape to urge fluids contained or deposited into the bioreactor vial 13000 to flow into and collect in the reactor well 13070. Reactor well 13070 is configured to have a size and shape to closely match the size and shape of MSPM scaffolding 3000 received therein.

The top shell 13010 includes an inlet port 13100 and a drain port 13110 extending there through. While plugs 13120 are shown, during the process, plugs are not installed until the end of the process as will be described in detail below. As described above, when fluid is added to the bioreactor vial 13000, the interface tube 5150 is depressed such that the injection seal 9160 is seated against the inlet port 13100 and fluid is then pumped via lumen 9180 into the bioreactor vial 13000. The drain port 13110 includes a drain tube 13130 that extends to the bottom wall 13060 of the bioreactor vial 13000. When a vacuum is applied via lumen 9180 fluid is drawn out of the bioreactor vial 13000.

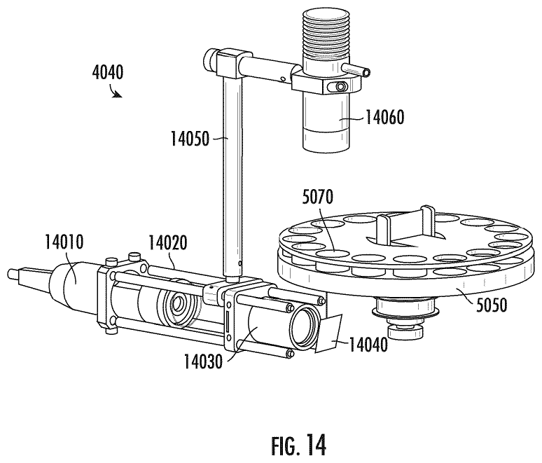

An imaging system 4040 is shown at FIG. 14. The imaging system 4040 is configured and arranged to illuminate the culture within the bioreactor vial 5070 and capture a high resolution image thereof in order to monitor the quality and consistency of the growth of the RPE culture on the MSPM scaffold contained therein. A camera 14010 is mounted in an adjustable framework 14020 that includes a lens array 14030 on an opposing end thereof. In some arrangements the camera 14010 and lens array 14030 are positioned beneath the bioreactor vial 5070. In some arrangements the camera 14010 and lens array 14030 are arranged orthogonally relative to the bioreactor vial 5070 and a mirror 14040 is employed to direct and image the bioreactor vial 5070 to the camera 14010 and lens array 14030. Additionally, a support 14050 extends upwardly and supports a lighting source 14060 to illuminate the culture within the bioreactor vial 5070. It should be appreciated that the position of the camera 14010 and lens array 14030 could be axial relative to the bioreactor vial 5070. In other arrangements, the locations of the camera 14010 and lens array 14030 could be reversed with the lighting source 14060 such that the contents of the bioreactor vial 5070 are imaged directly.

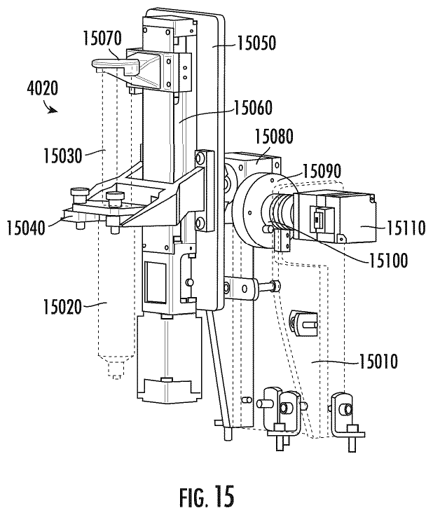

FIG. 15 provides a general illustration of a fluid delivery system 4020 in accordance with an aspect of the present disclosure. The fluid delivery 4020 system includes a mounting stanchion 15010 that supports the fluid delivery system 4020 relative to the upper base 5030. The fluid delivery system 4020 comprises a syringe pump supporting the barrel of a syringe 15020 with a plunger 15030 received therein. The syringe barrel 15020 is received and retained in a retainer bracket 15040 that is in turn mounted on agitator plate 15050. In addition, an actuator 15060 is coupled to the agitator plate 15050 having an actuator slide 15070 that is linearly translatable along the actuator 15060. The actuator slide 15070 is engaged with a top end of the plunger 15030. Displacement of the actuator slide 15070 along the actuator 15060 depresses the plunger 15030 relative to the syringe barrel 15020 causing the syringe pump 15020 to dispense a measured quantity of the contents therein. Depending on the materials being dispensed and the step in the process, the contents of the syringe pump 15020 may include Dulbecco's Phosphate Buffered Saline (DPBS), a solution of viable RPE cells, growth media, vitronectin, cryo preservative and combinations thereof. As each fluid is to be delivered, syringe pump 15020 is exchanged with an alternate syringe barrel 15020 containing the desired fluid for dispensing.

It is known in the art that RPE cells are extremely fragile and prone to clumping. To maintain the RPE cells in solution in preparation for dispensing, the agitator plate 15050 is mounted to a rotational agitator 15080 that slowly rotates back and forth to gently rock the syringe pump 15020 thereby agitating the contents within the syringe pump 15020. The speed and frequency of the motion are carefully controlled to prevent over agitation and damage to the RPE cells.

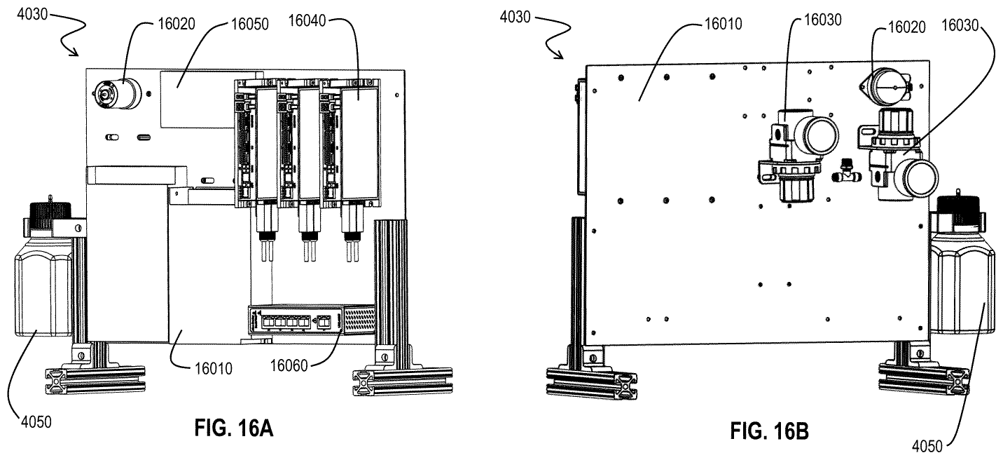

An exemplary controller 4030 is generally and schematically illustrated at FIGS. 16A-16B. A waste container 4050 is shown mounted on the backer 16010, although this position is shown merely for convenience as the waste container 4050 may be positioned in any of a variety of locations within the system and method of the present disclosure. A suction pump 16020, such as a peristaltic pump, is mounted to the controller 4030 to create the suction needed to withdraw fluids from the bioreactor vials as needed during the process. Further, there is an air supply either in the form of a facility dedicated air system or alternately an air pump to supply controlled pressurized air via regulators 16030 to drive the pneumatically driven and controlled elements of the method and system of the disclosure. Similarly, motor controllers 16040 are mounted to the backer 16010 to provide control signal and power to the various motors employed within the method and system. A processor 16050, such as for example a programmable logic controller (PLC), is seen to be in electrical communication with the various sensors, systems, motor controllers and pumps within the system to provide instructions controlling the various elements of the system, while also receiving and processing feedback to adjust operating parameters based on sensor or user inputs. In addition, a communications bus 16060 is provided to allow network connectivity as well as remote commands and external instructions to be transferred to/from the controller 4030.

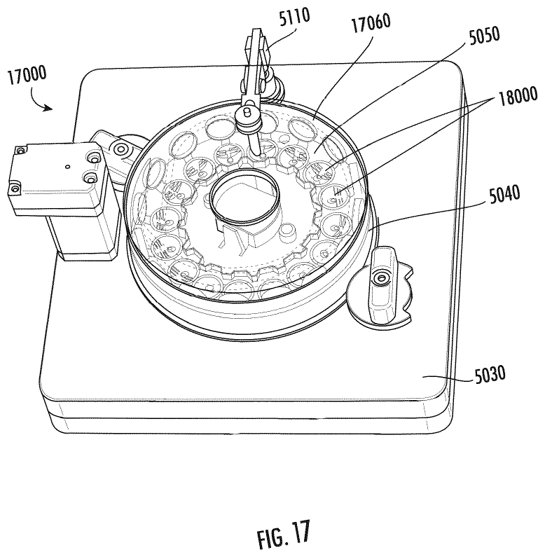

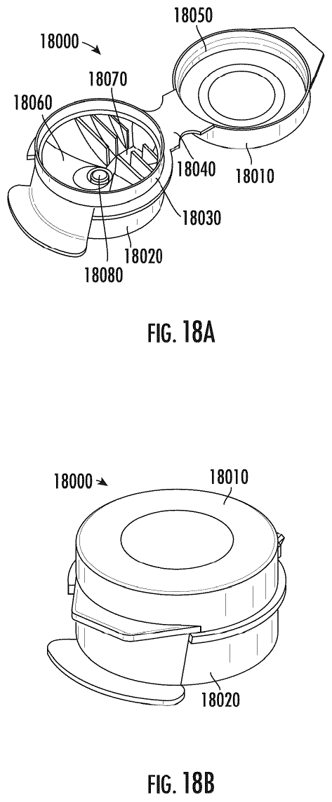

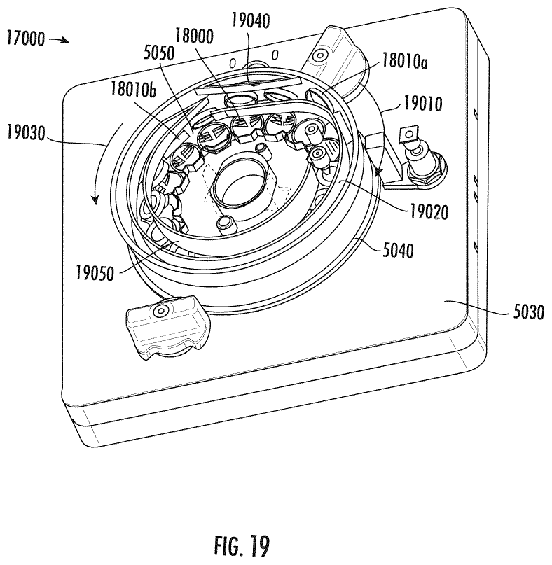

FIGS. 17-19 provide an alternate arrangement system suitable for implementation within the teachings of the present disclosure. In all aspects, the alternate system 17000 operates as disclosed above with the following alternate arrangements. The system of this embodiment employ a one-piece bioreactor vial 18000 that will be described at FIG. 18 in more detail below. The shuttle carrier 5050 receives and retains at least one of a plurality of bioreactor vials 18000 and provides a rotational motion of the bioreactor vials 18000 relative to the operable components on the system. Only one of the spring biased actuator arms 5090 is shown as at least one input arm 5110 which is mounted to the upper base 5020. As stated above, the shuttle carrier 5050 is received within the shuttle table 5040 such that the shuttle table 5040 guides the motion of the shuttle carrier 5050. The lid 17060 has an alternate configuration and includes functional elements that will be further described at FIG. 19 below.

As can be seen at, FIGS. 18A-18B one configuration of an alternate bioreactor vial 18000 is shown suitable for use in accordance with the teachings herein. The bioreactor 18000 comprises a top shell 18010 and a bottom shell 18020 thereby forming a one-piece bioreactor vial 18000. In this arrangement the top shell 18010 is affixed to the bottom shell 18020 via a hinge element 18040. The bottom shell 18020 includes an upwardly extending side wall 18030 that receives and seals against a downwardly extending sidewall 18050 on the top shell 18010 when the top shell 18010 and bottom shell 18020 are received in assembled relation with one another. A bottom wall 18060 is shaped to have a downwardly tapered shape to urge fluids contained or deposited into the bioreactor vial 18000 to flow into and collect in the reactor well 18070 and around the vial drain 18080. Reactor well 18070 is configured to have a size and shape to closely match the size and shape of MSPM scaffolding received therein. Fins rise from the bottom wall 18060 adjacent the reactor well 18070. The fins are fluid-dynamically engineered so as to retain small amounts of fluid trapped there between when fluid is drawn out of the bioreactor vial 18000 to allow enough fluid to remain and drain back into the reactor well 18070 to ensure that the MSPM does not dry out during the various stages of the process disclosed herein. Further, the fins also limit the movement of the MSPM as within the reactor well 18070.

The top shell 18010 remains in an open position as shown at FIG. 18A so that fluids can be added to and directly removed from the bioreactor vial 18000 without the need for inlet and outlet ports. In preparation for transplantation, the user inserts a pin into vial drain 18080 to puncture the vial drain 18080 thereby allowing preservative fluid contained within the bioreactor vial 18000 to drain therefrom. In an alternate configuration a pull tab may be provided on the bottom of the bioreactor vial 18000 that when pulled, removes the vial drain 18080.

Turning now to FIG. 19, the lid 17060 of the present embodiment provides additional functionality as stated above. With the bioreactor vials 18000 contained in the shuttle carrier 5050 rotating in a first, processing direction, the top shell 18010a, can be seen to remain in an open position relative to the bioreactor vial 18000. In this case, as can be seen with shuttle carrier 5050 rotating in a clockwise direction as shown by arrow 19010, the top shells 18010a are held in an upright position within the process channel 19020. Alternately, when the process reaches its conclusion, the shuttle carrier is rotated in an anti-clockwise direction as shown by arrow 19030. When rotation in anti-clockwise direction 19030, the top shells are 18010 displaced inwardly by the bumper 19040 flexing hinge 18040. Continuing this rotation, the top shells 18010b encounter closing bar 19050 that captures the top shell 18010b there beneath. The closing bar has a tapered lower edge that urges the top shell 18010b downwardly into a closed position on bottom shell 18020 as rotation continues. It should be appreciated that directional rotations were specified, the bumper 19040 and closing bar 19050 structures could be reversed to accommodate rotations in the opposite directions. Further, the bumper 19040 and closing bar 19050 structures may be formed as integral features on the interior of the lid 17060, may be an insert of its own or may be freestanding structures.

In accordance with a method in accordance with an embodiment of the present disclosure, at least one bioreactor vial is provided that is configured to be supported and received by an automated handling and processing system. The bioreactor vial has a reactor well therein into which an MSPM scaffold is received. Various support fluids are added and subsequently RPE cells are seeded onto the MSPM. The RPE cells form a culture of monolayer of hexagonally shaped RPE cells that is adhered to the MSPM which is suitable for subsequent transplantation into an eye in order to develop in a manner that supports and maintains the photoreceptors of the retina.

In an alternate method, the present disclosure teaches a method whereby one or more bioreactor vials are provided, wherein each bioreactor vial is configured to be supported and received by an automated handling and processing system. The bioreactor vial has a reactor well therein into which an MSPM scaffold is received. Various support fluids are added and subsequently RPE cells are seeded onto the MSPM. The RPE cells form a culture of monolayer of hexagonally shaped RPE cells that is adhered to the MSPM which is suitable for subsequent transplantation into an eye in order to develop in a manner that supports and maintains the photoreceptors of the retina.

In a further alternate method, the present disclosure teaches a method whereby a plurality of MSPM scaffolds are individually positioned in a reactor well within each of a plurality of bioreactor vials, the bioreactor wells each configured to securely receive and retain the MSPM scaffold received therein. A fluid delivery system transfers required process fluids into and out of the reactor well in a manner that reduces damage as well as potential contamination to the tissue culture during growth, handling, transport and preparation for transplantation.

In yet a further method, the present disclosure teaches a method for automated production of a viable cell culture supported on a mesh supported membrane lattice that is suitable for therapeutic implantation in connection with regenerative cell therapy wherein a shuttle system having a plurality of bioreactor stations is provided wherein each bioreactor station is configured to receive and retain a bioreactor vial therein. The shuttle system is moved in a manner that each station is moved to position the bioreactor vial adjacent to one or more material delivery stations that serve to position an MSPM scaffold in each of the reactor wells within each of a plurality of bioreactor vials, deliver and remove various fluids, seed cells onto the MSPM scaffolds, deliver cryogenic preservative fluid and/or cap or seal the bioreactor vials in a manner that reduces damage as well as potential contamination to the tissue culture during growth, handling, transport and preparation for transplantation.

In some configurations method includes steps wherein the bioreactor vials are sealed and accessed via ports therein. In some configurations, the bioreactor vials have a displaceable cap that remains in an open position until the final step in processing.

In some configurations, the reactor well in each of the bioreactor vials include elements that direct and control fluid movement within the reactor well to prevent dislodging, floating, crumpling, creasing and/or inversion of the MSPM scaffold contained therein.

In some configurations, the shuttle system is in a closed environment.

In some configurations, the shuttle system has multiple tiers, each tier including a plurality of bioreactor stations thereon.

In other configurations, a camera is included to provide visualization of the MSPM scaffold contained within the reactor well of the bioreactor vials.

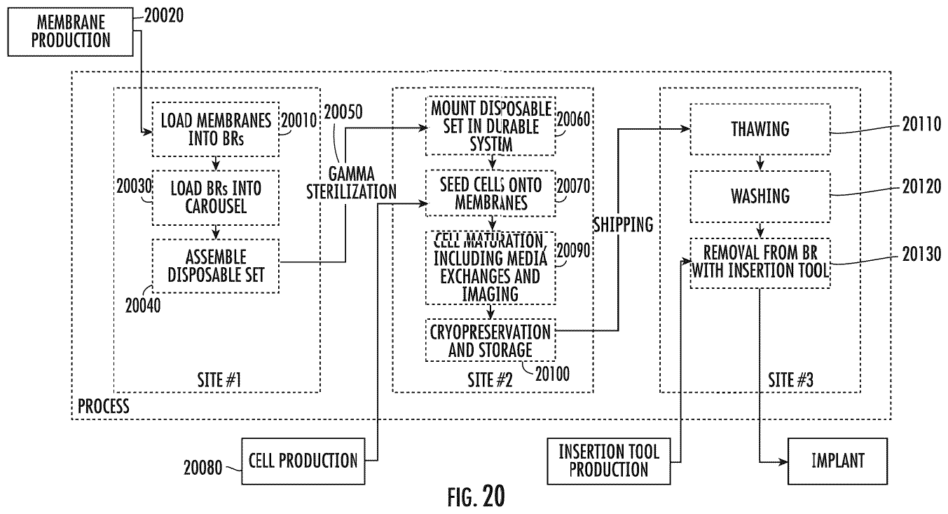

As illustrated in FIG. 20, In a further configuration in accordance with the present disclosure, a method is taught for automated production of a mesh supported membrane lattice for supporting cultured cells that is suitable for therapeutic implantation in connection with regenerative cell therapy. The system in the present disclosure is designed to be sterilized while partially assembled. The shuttle carrier is sterilized with bioreactor vials loaded therein. The shuttle carrier maintains a sterile barrier and prior to removal from the shuttle carrier each bioreactor vial is scaled to retain its own sterility until implantation. The syringes are sterile upon arrival. All parts of the process where the sterile barrier will be broken, such as media preparation, take place in a sterile biosafety cabinet (BSC) while following sterile practices.

Each of the individually consolidated bioreactor vials will provide a safe and sterile location for each MSPM scaffold to be seeded, matured, frozen, and shipped. The MSPM scaffolds do not need to be handled between initial placement and removal for surgery. They are designed to be compatible with the surgical transplantation tool for easy MSPM scaffold removal. The bioreactor vials easily fit in the palm of a hand, and have rigid edges that allow for easy opening and secure closing. Once the MSPM scaffold is removed, the bioreactor vial is disposed of.

Referring again to FIG. 20, the method of the present disclosure begins at with a preparation step prior to the beginning of the culture growth of the process. Bioreactor vials, filled with DPBS, are loaded with MSPM scaffolds 20010 and dried to cause the MSPM scaffolds to adhere to the reactor well within the bioreactor vial. In one configuration, MSPM scaffolds are manufactured at an alternate location 20020. Bioreactors, with MSPM scaffolds loaded therein, are installed into the shuttle carrier 20030 and the assembly of the shuttle carrier is completed 20040. The shuttle carrier with the MSPM scaffold loaded bioreactor vials is then sterilized 20050. After the sterilization step, the MSPM scaffolds will not be handled again until transplantation surgery.

The shuttle carrier with the MSPM scaffold loaded bioreactor vials is then installed into a system in accordance with the present disclosure and the entire system is placed into an incubator 20060, with incubator settings preferably set to a temperature of 37 C, 5% CO2 and 90% humidity. In the next part of the process, the MSPM scaffolds within each bioreactor vial may be imaged to identify any potentially damaged scaffolds. Then a solution of vitronectin and DBPS is prepared and added to each bioreactor vial in preparation for seeding a solution of cells onto the MSPM scaffolds within each bioreactor vial 20070 and the concentration of cells in each bioreactor is observed and adjusted to a predetermined level. In one configuration, production of cells for seeding is completed at an alternate location 20080.

During the culture growth phases 20090, growth media such as CTS AIM-V is added to the bioreactor vials and periodically exchanged while cell culture growth is monitored.

At the end of the culture growth phase, a controlled rate freezer (CRF) is prepared and spent media is drained from the bioreactor vials containing cultures and transferred to a waste container for quality control testing. On the seventh day of the process the timing is critical to ensure the cryoprotectant does not cause any damage to the cells. The automated process will ensure that this series of steps will be done in the same interval of time, preferably in 20 minutes or less on every run. Cryoprotectant CS10 is added to each of the bioreactor vials 20100 and each of the bioreactor vials is sealed. And the relevant disposables, the shuttle carrier and bioreactor vials, are placed into the controlled rate freezer (CRF) to ensure that reusable components do not get damaged from extreme cold temperatures.

Upon completion of the CRF cycle, the bioreactors will be transferred for storage until surgery. The shuttle carrier is opened and the bioreactor vials are transferred to a storage freezer. Prior to surgical transplantation, a single bioreactor vial is removed from the freezer and thawed 20110. The surgeon drains the cryoprotectant from the bioreactor vial, rinses the residual cryoprotectant, opens the bioreactor vial and inspects the MSPM scaffold in preparation for transplantation 20120. The surgeon then loads the MSPM scaffolding into the transplantation tool for transplantation 20130. While FIG. 20 depicts the process being performed at 3 different sites, one skilled in the art can appreciate that various steps of the process may all be performed at the same site or various steps may be shifted from one site to another and still fall within the scope of the invention.

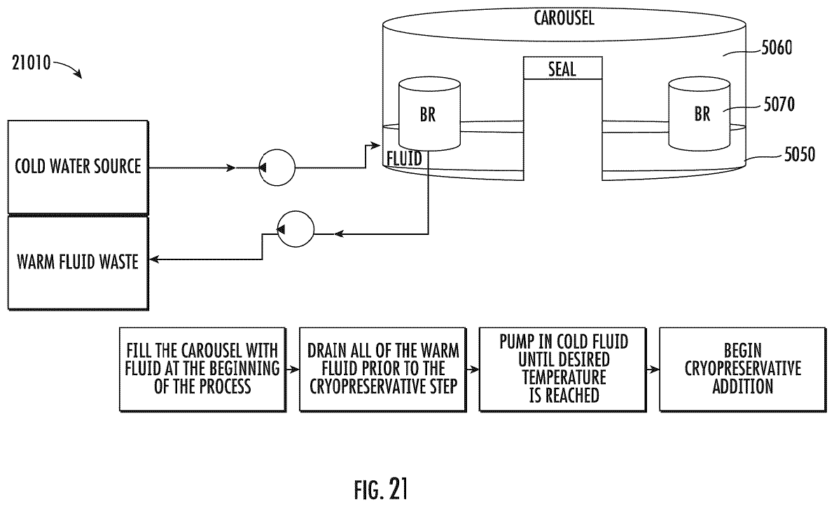

Turning now to FIG. 21 a schematic diagram is provided to illustrate a method and system of establishing thermal management of the bioreactor vials 5070 supported in the carousel consisting of the lid 5060 and the shuttle carrier 5050. A thermal management system 21010 may be fluidically connected to the carousel assembly such that a fluid is deposited into a well on the top of the shuttle carrier 5050. The well on the shuttle carrier 5050 is filled with fluid at the beginning of the culturing process such that the fluid provides both thermal mass to maintain a consistent temperature of the bioreactor vial 5070 as well as to provide a means for controlling the humidity of the cavity in the carousel as between the shuttle carrier 5050 and the lid 5060 to maintain a constant humidity in the bioreactor vials 5070. The initial fluid loading remains in place during the culture growing process. Once the cultures within the bioreactor vials 5070 is grown, in preparation for the cryopreservation step, the fluid is removed to a fluid waste container. A cold fluid is then added to the well on the shuttle carrier 5050. The addition of the cold fluid serves to precool the thermal mass of the bioreactor vial 5070 and the contents therein. In this manner, the precooling of the culture within the bioreactor vial 5070 then allows the addition of cryo preservative in a manner that rapidly freezes the culture while minimizing the potential of damage to the culture during this preservation step.

Accordingly, the present disclosure provides a method and system for automated production of a viable cell culture on a mesh supported membrane lattice that is suitable for therapeutic implantation in connection with regenerative cell therapy. It is a further object of the present disclosure to provide a method and system that provides maximum throughput and efficient production of a mesh supported membrane lattice for supporting cultured cells that is suitable for therapeutic implantation in connection with regenerative cell therapy. It is still a further object of the present disclosure to provide a method and system for automated production of a mesh supported membrane lattice for supporting cultured cells that is suitable for therapeutic implantation in connection with regenerative cell therapy that reduces damage as well as potential contamination to the tissue culture during growth, handling, transport and preparation for transplantation. It is a still further object of the disclosure to provide a method and system for automated production of a mesh supported membrane lattice for supporting cultured cells that is suitable for therapeutic implantation in connection with regenerative cell therapy that improves the efficiency with which the quantities of viable RPE cells, growth media and vitronectin are consumed in producing the completed MSPM scaffolding supporting a culture of monolayer of hexagonally shaped RPE cells.

In some aspects of the method and system of the present disclosure, some components are single use, disposable parts such as, bioreactor vials, syringes, tubing sets and waste containers. Other components are durable reusable components such as the shuttle system, an enclosure, pumping subsystem and the imaging subsystem. The use of a combination of disposable and durable parts ensures sterility and maximizes product yield during implementation of the automated process.

These together with other objects of the disclosure, along with various features of novelty which characterize the method and system of the present disclosure, are pointed out with particularity in the claims annexed hereto and forming a part of this disclosure. For a better understanding of the method and system of the disclosure, its operating advantages and the specific objects attained by its uses, reference should be had to the accompanying drawings and descriptive matter in which there are illustrated various embodiments of the method and system disclosed herein.

Various alternatives and modifications can be devised by those skilled in the art without departing from the disclosure. Accordingly, the present disclosure is intended to embrace all such alternatives, modifications and variances. Additionally, while several example configurations of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular configurations. In addition, those skilled in the art will envision other modifications within the scope and spirit of the claims appended hereto. Other elements, steps, methods and techniques that are insubstantially different from those described above and/or in the appended claims are also intended to be within the scope of the disclosure.

The drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the∞, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B∞ should not be limited to devices consisting only of components A and B.

Furthermore, the terms “first”, “second”, “third,” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the example configurations of the disclosure described herein are capable of operation in other sequences and/or arrangements than are described or illustrated herein.

What is claimed is:

1. A system for developing a viable culture of retinal pigment epithelial (RPE) cells on a mesh supported membrane, comprising:
at least one bioreactor vial;
an automated handling system that receives said at least one bioreactor vial;
a fluid delivery system for adding and removing support fluids within said at least one bioreactor vial; and
a controller for directing and controlling operation of said system.

2. The system of claim 1, said at least one bioreactor vial further comprising:
a plurality of bioreactor vials.

3. The system of claim 1, further comprising:
a thermal control system to monitor a temperature of said at least one bioreactor vial and to maintain said at least one bioreactor vial at a predetermined temperature.

4. The system of claim 1, further comprising:
an imaging system to observe and monitor development of said RPE cell culture within said at least one bioreactor vial.

5. The system of claim 1, the at least one bioreactor vial further comprising:
a bottom shell having side walls and a tapered bottom wall, said bottom wall forming a bioreactor well for receiving a mesh supported membrane scaffold therein; and
a top shell configured to be received in sealed, mating relation with said bottom shell.

6. The system of claim 5, further comprising:
at least one port in said top shell to allow access to an interior of said at least one bioreactor vial.

7. The system of claim 5, further comprising:
at least one access port in said top shell to allow exchange of support fluids to and from an interior of said at least one bioreactor vial; and
at least one vent port in said top shell to allow exchange of gas to and from an interior of said at least one bioreactor vial.

8. The system of claim 5, further comprising:
a vial drain positioned at a low point in said tapered bottom wall.

9. The system of claim 5, further comprising:
fins extending upwardly from said tapered bottom wall adjacent said bioreactor well.

10. The system of claim 9, said fins cooperating with said bioreactor well to maintain a position of said mesh supported membrane scaffold contained therein.

11. The system of claim 5, said bioreactor well configured to have a size and shape to maintain a position of said mesh supported membrane scaffold contained therein.

12. The system of claim 1, the at least one bioreactor vial further comprising:
a bottom shell having side walls and a tapered bottom wall, said bottom wall forming a bioreactor well for receiving a mesh supported membrane scaffold therein; and
a top shell attached to said bottom shell via a hinge, said top shell configured to be received in sealed, mating relation with said bottom shell.

13. The system of claim 12, the top shell having an open position and a closed position wherein said top shell is received in sealed, mating relation with said bottom shell.

14. The system of claim 13, wherein said top shell, in said open position allows addition of support fluids to an interior of said at least one bioreactor vial, and removal of support fluids from an interior of said at least one bioreactor vial.

15. The system of claim 12, further comprising:
a vial drain positioned at a low point in said tapered bottom wall.

16. The system of claim 12, further comprising:
fins extending upwardly from said tapered bottom wall adjacent said bioreactor well.

17. The system of claim 16, said fins cooperating with said bioreactor well to maintain a position of said mesh supported membrane scaffold contained therein.

18. The system of claim 12, said bioreactor well configured to have a size and shape to maintain a position of said mesh supported membrane scaffold contained therein.

19. The system of claim 1, the automated handling system, further comprising:
a base;
a shuttle table supported on a top surface of said base, said shuttle table including a recess therein; and
a shuttle carrier movably supported in said recess in said shuttle table, said shuttle table having a plurality of bioreactor wells therein, each configured to receive and support a bioreactor vial.

20. The system of claim 19, further comprising:
shuttle table locks configured to releasably engage said shuttle table to said base.

21. The system of claim 19, wherein said shuttle carrier moves within said shuttle table.

22. The system of claim 21, wherein a movement of said shuttle carrier is rotational.

23. The system of claim 21, wherein a movement of said shuttle carrier is linear.

24. The system of claim 19, further comprising: a
lid that is configured to mate with an upper rim of said shuttle table, cooperating to form a sealed environment around said shuttle carrier and said plurality of bioreactor vials contained therein.

25. The system of claim 24, said bioreactor vials further comprising:
a bottom shell having side walls and a tapered bottom wall, said bottom wall forming a bioreactor well for receiving a mesh supported membrane scaffold therein; and
a top shell attached to said bottom shell via a hinge, said top shell configured to be received in sealed, mating relation with said bottom shell,
the top shell having an open position and a closed position wherein said top shell is received in sealed, mating relation with said bottom shell

26. The system of claim 25, said lid further comprising:
formations on an inner surface of said lid,
wherein said formations urging said top shell to remain in said open position when said shuttle carrier is moved in a first direction,
wherein said formations urging said top shell to said closed position when said shuttle carrier is moved in a second direction opposite said first direction.

27. The system of claim 26, wherein said formations are integrally formed with said lid.

28. The system of claim 26, wherein said formations are an insert received within said lid.

29. The system of claim 19, further comprising:
a stepper motor engaged with said shuttle table and said controller,
wherein said stepper motor causes motion of said shuttle table in response to instructions from said controller.

30. The system of claim 4, said imaging system further comprising:
a camera positioned adjacent a first end of said bioreactor vial; and
an illumination source positioned adjacent a second end of said bioreactor vial.

31. The system of claim 30, wherein said camera is above said bioreactor vial and said illumination source is below said bioreactor vial.

32. The system of claim 31, further comprising:
illumination source to said bioreactor vial.

33. The system of claim 30, wherein said camera is below said bioreactor vial and said illumination source is above said bioreactor vial.

34. The system of claim 33, further comprising:
a mirror below said bioreactor vial, said mirror directing an image from said bioreactor vial to said camera.

35. The system of claim 1, the fluid delivery system further comprising:
at least one actuator arm, said actuator arm being spring biased to a normally retracted position.

36. The system of claim 35, the fluid delivery system further comprising:
an actuator to move said at least one actuator arm to an engaged position relative to said at least one bioreactor vial in response to a signal from said controller.

37. The system of claim 36, further comprising:
a syringe pump supporting a syringe barrel with a plunger received therein, said syringe pump including an actuator slide in engagement with said plunger,
wherein said syringe pump displaces said actuator slide in response to instructions from said controller to displace said plunger.

38. The system of claim 37, wherein portions of said syringe pump comprise durable components and portions of said syringe pump comprise disposable components.

39. The system of claim 38, wherein said syringe barrel and said plunger are disposable.

40. The system of claim 37, wherein said syringe pump is in fluid communication with a delivery tube,
wherein displacement of said plunger causes fluid to be dispensed via said delivery tube into said bioreactor vial.

41. The system of claim 37, further comprising:
an agitator plate supporting said syringe pump, said agitator plate rocking said syringe pump to mix contents of said syringe barrel maintaining said contents in a uniform solution.

42. The system of claim 41, wherein portions of said syringe pump comprise durable components and portions of said syringe pump comprise disposable components.

43. The system of claim 42, wherein said syringe pump, said actuator slide and said agitator plate are durable.

44. The system of claim 35, the at least one actuator arm further comprising:
one input actuator arm, said input actuator arm being spring biased to a normally retracted position; and
one drain actuator arm, said drain actuator arm being spring biased to a normally retracted position

45. The system of claim 44, the fluid delivery system further comprising:
a first actuator to move said input actuator arm to an engaged position in contact with said at least one bioreactor vial in response to a signal from said controller; and
a second actuator to move said drain actuator arm to an engaged position in contact with said at least one bioreactor vial in response to a signal from said controller.

46. The system of claim 4, wherein the automated handling system, the imaging system and the controller are durable.

47. The system of claim 4, wherein the at least one bioreactor vial is a single use disposable.

48. A method for developing a viable culture of retinal pigment epithelial (RPE) cells on a mesh supported membrane, comprising:
providing at least one bioreactor vial positioned within an automated handling system;
positioning a mesh supported membrane scaffold within said at least one bioreactor vial;
seeding said mesh supported membrane scaffold with said RPE cells;
adding and removing support fluids within said at least one bioreactor vial; and
monitoring and controlling growth of said RPE cells via a controller

49. The method of claim 48, said at least one bioreactor vial further comprising:
a plurality of bioreactor vials.

50. The method of claim 48, further comprising:
monitoring and maintaining a temperature of said at least one bioreactor vial at a predetermined temperature using a thermal control system.

51. The method of claim 48, further comprising:
observing and monitoring development of said RPE cell culture within said at least one bioreactor vial using an imaging system.

52. The method of claim 48, the at least one bioreactor vial further comprising:
a bottom shell having side walls and a tapered bottom wall, said bottom wall forming a bioreactor well for receiving a mesh supported membrane scaffold therein; and
a top shell configured to be received in sealed, mating relation with said bottom shell.

53. The method of claim 52, further comprising:
at least one port in said top shell to allow access to an interior of said at least one bioreactor vial.

54. The method of claim 52, further comprising:
at least one access port in said top shell to allow exchange of support fluids to and from an interior of said at least one bioreactor vial; and
at least one vent port in said top shell to allow exchange of gas to and from an interior of said at least one bioreactor vial.

55. The method of claim 52, further comprising:
a vial drain positioned at a low point in said tapered bottom wall.

56. The method of claim 52, further comprising:
fins extending upwardly from said tapered bottom wall adjacent said bioreactor well.

57. The method of claim 56, said fins cooperating with said bioreactor well to maintain a position of said mesh supported membrane scaffold contained therein.

58. The method of claim 52, said bioreactor well configured to have a size and shape to maintain a position of said mesh supported membrane scaffold contained therein.

59. The method of claim 48, the at least one bioreactor vial further comprising:
a bottom shell having side walls and a tapered bottom wall, said bottom wall forming a bioreactor well for receiving a mesh supported membrane scaffold therein; and
a top shell attached to said bottom shell via a hinge, said top shell configured to be received in sealed, mating relation with said bottom shell.

60. The method of claim 59, the top shell having an open position and a closed position wherein said top shell is received in sealed, mating relation with said bottom shell.

61. The method of claim 60, wherein said top shell, in said open position allows addition of support fluids to an interior of said at least one bioreactor vial, and removal of support fluids from an interior of said at least one bioreactor vial.

62. The method of claim 59, further comprising:
a vial drain positioned at a low point in said tapered bottom wall.

63. The method of claim 59, further comprising:
fins extending upwardly from said tapered bottom wall adjacent said bioreactor well.

64. The method of claim 63, said fins cooperating with said bioreactor well to maintain a position of said mesh supported membrane scaffold contained therein.

65. The method of claim 59, said bioreactor well configured to have a size and shape to maintain a position of said mesh supported membrane scaffold contained therein.

66. The method of claim 48, the automated handling system, further comprising:
a base;
a shuttle table supported on a top surface of said base, said shuttle table including a recess therein; and
a shuttle carrier movably supported in said recess in said shuttle table, said shuttle table having a plurality of bioreactor wells therein, each configured to receive and support a bioreactor vial.

67. The method of claim 66, further comprising:
shuttle table locks configured to releasably engage said shuttle table to said base.

68. The method of claim 66, wherein said shuttle carrier moves within said shuttle table.

69. The method of claim 68, wherein a movement of said shuttle carrier is rotational.

70. The method of claim 68, wherein a movement of said shuttle carrier is linear.

71. The method of claim 66, further comprising:
a lid that is configured to mate with an upper rim of said shuttle table, cooperating to form a sealed environment around said shuttle carrier and said plurality of bioreactor vials contained therein.

72. The method of claim 71, said bioreactor vials further comprising:
a bottom shell having side walls and a tapered bottom wall, said bottom wall forming a bioreactor well for receiving a mesh supported membrane scaffold therein; and
a top shell attached to said bottom shell via a hinge, said top shell configured to be received in sealed, mating relation with said bottom shell,
the top shell having an open position and a closed position wherein said top shell is received in sealed, mating relation with said bottom shell

73. The method of claim 72, said lid further comprising:
formations on an inner surface of said lid,
wherein said formations urging said top shell to remain in said open position when said shuttle carrier is moved in a first direction,
wherein said formations urging said top shell to said closed position when said shuttle carrier is moved in a second direction opposite said first direction.

74. The method of claim 73, wherein said formations are integrally formed with said lid.

75. The method of claim 73, wherein said formations are an insert received within said lid.

76. The method of claim 66, further comprising:
a stepper motor engaged with said shuttle table and said controller,
wherein said stepper motor causes motion of said shuttle table in response to instructions from said controller.

77. The method of claim 51, said imaging system further comprising:
a camera positioned adjacent a first end of said bioreactor vial; and
an illumination source positioned adjacent a second end of said bioreactor vial.

78. The method of claim 77, wherein said camera is above said bioreactor vial and said illumination source is below said bioreactor vial.

79. The method of claim 78, further comprising:
a mirror below said bioreactor vial, said mirror directing illumination from said illumination source to said bioreactor vial.

80. The method of claim 77, wherein said camera is below said bioreactor vial and said illumination source is above said bioreactor vial.

81. The method of claim 80, further comprising:
a mirror below said bioreactor vial, said mirror directing an image from said bioreactor vial to said camera.

82. The method of claim 48, the fluid delivery system further comprising:
at least one actuator arm, said actuator arm being spring biased to a normally retracted position.

83. The method of claim 82, the fluid delivery system further comprising:
an actuator to move said at least one actuator arm to an engaged position in contact with said at least one bioreactor vial in response to a signal from said controller.

84. The method of claim 83, further comprising:
a syringe pump supporting a syringe barrel with a plunger received therein, said syringe pump including an actuator slide in engagement with said plunger,
wherein said syringe pump displaces said actuator slide in response to instructions from said controller to displace said plunger.

85. The method of claim 84, wherein portions of said syringe pump comprise durable components and portions of said syringe pump comprise disposable components.

86. The method of claim 85, wherein said syringe barrel and said plunger are disposable.

87. The method of claim 83, wherein said syringe pump is in fluid communication with said actuator arm,
wherein displacement of said plunger causes fluid to be dispensed via said actuator arm into said bioreactor vial.

88. The method of claim 83, further comprising:
an agitator plate supporting said syringe pump, said agitator plate rocking said syringe pump to mix contents of said syringe barrel maintaining said contents in a uniform solution.

89. The method of claim 88, wherein portions of said syringe pump comprise durable components and portions of said syringe pump comprise disposable components.

90. The method of claim 89, wherein said syringe pump, said actuator slide and said agitator plate are durable.

91. The method of claim 84, the at least one actuator arm further comprising:
one input actuator arm, said input actuator arm being spring biased to a normally retracted position; and
one drain actuator arm, said drain actuator arm being spring biased to a normally retracted position

92. The method of claim 91, the fluid delivery system further comprising:
a first actuator to move said input actuator arm to an engaged position in contact with said at least one bioreactor vial in response to a signal from said controller; and
a second actuator to move said drain actuator arm to an engaged position in contact with said at least one bioreactor vial in response to a signal from said controller.

93. The method of claim 51, wherein the automated handling system, the imaging system and the controller are durable.

94. The method of claim 51, wherein the at least one bioreactor vial is a single use disposable.

95. The method of claim 48, wherein the at least one bioreactor vial is utilized for culture growth, preservation, storage and transportation of said RPE cell culture.

96. The system of claim 1, wherein the at least one bioreactor vial is utilized for culture growth, preservation, storage and transportation of said RPE cell culture.
